(19) United States
(12) Patent Application Publication
(10) Pub. No.: US 2013/0245396 A1
(43) Pub. Date: Sep. 19, 2013

(54) MENTAL STATE ANALYSIS USING WEARABLE-CAMERA DEVICES

(71) Applicant: AFFECTIVA, INC., Waltham, MA (US)

(72) Inventors: David Berman, San Jose, CA (US); Rana el Kaliouby, Waltham, MA (US); Rosalind Wright Picard, Newtonville, MA (US)

(73) Assignee: Affectiva, Inc., Waltham, MA (US)

(21) Appl. No.: 13/886,249

(22) Filed: May 2, 2013

Related U.S. Application Data
(63) Continuation-in-part of application No. 13/153,745, filed on Jun. 6, 2011.
(60) Provisional application No. 61/641,852, filed on May 2, 2012.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.

(51) Int. Cl. A61B 5/16

(52) U.S. Cl. CPC A61B 5/165
USPC 600/301

(57) ABSTRACT

Mental state analysis may be performed using a wearable-camera device. Embodiments provide a glasses mounted camera or an ear-mounted device comprising a camera to collect mental state data of an individual being viewed. Information about the mental states of the individual being viewed can be fed back to the individual wearing the wearable-camera device via visual, verbal, or tonal indicators. Various emotional indicators can be provided to the wearer of the device. Analysis of the mental state data of the person being observed can be performed on the wearable-camera device, on a mobile platform, on a server, or a combination thereof. Shared and aggregated mental state information may be shared via social networking. A geographical representation of the mental state information may be rendered.

### RELATED APPLICATIONS

This application claims the benefit of U.S. provisional patent application “Ear-Mounted Mental State Analysis Device” Ser. No. 61/641,852, filed May 2, 2012. This application is also a continuation-in-part of U.S. patent application “Mental State Analysis Using Web Services” Ser. No. 13/153,745, filed Jun. 6, 2011 which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Data Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011. The foregoing applications are hereby incorporated by reference in their entirety.

### FIELD OF ART

This application relates generally to emotion analysis and more particularly to a mental state analysis using a wearable-camera device.

### BACKGROUND

People spend a tremendous amount of time engaged in interaction with one another. Perceiving another's emotional state is critical to successful interaction with that person. A person may be happy, confident, confused, frustrated, smiling, or frowning and these states can directly impact interaction with that person. If, however, an individual interacting this person is not able to pick up on the various cues which indicate the emotions of the person being viewed, the interaction can become problematic. Therefore, evaluation of the mental states of a person being viewed is exceedingly important to effective human interaction. It is understood that a mental state can be an emotional or cognitive state and can be a mental response unconsciously triggered by the brain. In addition, an associated physical response can accompany an emotion (e.g. increased heart rate).

### SUMMARY

Analysis of people, as they interact with other people or with various forms of media, may be performed by gathering mental states through the evaluation of facial expressions, head gestures, and physiological conditions. In some cases, people, such as those with autism-spectrum disorders or with sight limitations, may have trouble recognizing the mental state of someone with whom they are interacting. Such people may not recognize confusion, anger, or other mental states in another person. A wearable device can provide analysis of a subject being viewed by the wearer, and provide that analysis to the wearer. The subject, or person being observed (PBO), may be analyzed, and the mental state information of the PBO rendered to the viewer wearing the apparatus—referred to herein as the device-wearing person (DWP). In other applications, the device may also collect information about DWPs, such as heart rate, body temperature, and other physical parameters. The mental state analysis of the DWP and/or the PBO may be uploaded to a server for additional analysis and rendering. The uploaded information may be sent to a social media site and rendered in a map format. A computer-implemented method for mental state analysis is disclosed comprising: collecting mental state data using a wearable-camera device wherein the wearable-camera device includes an ear-mounted camera; analyzing the mental state data to produce mental state information; and rendering the mental state information.

The wearable-camera device may include one or more of an ear-mounted camera, a glasses-mounted camera, a shoulder-mounted camera, or a clothing-mounted camera. The mental state data may be collected on a person at whom the wearable-camera device is pointed. The wearable-camera device may be on a wearer, where the wearer's head is pointed at the person at whom the wearable-camera device is pointed. The rendering may produce audio feedback on the mental state information. The audio feedback may be provided to a wearer of the wearable-camera device. The wearer of the wearable-camera device may be visually impaired. The wearer of the wearable-camera device may have a non-verbal learning disorder. The wearer of the wearable-camera device may be autistic. The rendering may include a display of mental state information. The collecting mental state data may further comprise collecting physiological data including one of electrodermal activity, heart rate, heart rate variability, skin temperature, and respiration. The collecting of physiological data may be accomplished using a sensor that is mounted on a person on whom the mental state data is being collected. The collecting of mental state data may further comprise actigraphy data. The method may further comprise storing mental state information based on the mental state data which was collected. The mental state information may be transmitted to a mobile platform. The mobile platform may be one of a mobile phone, a tablet computer, or a mobile device. The mental state information may be transmitted from the mobile platform to a server. The method may further comprise receiving mental state analysis from a server based on the mental state information. The rendering may be based on the mental state analysis received from the server. The method may further comprise inferring mental states based on the mental state data which was obtained wherein the mental states include one or more of frustration, confusion, disappointment, hesitation, cognitive overload, focusing, engagement, attention, boredom, exploration, confidence, trust, delight, disgust, skepticism, doubt, satisfaction, excitement, laughter, calmness, stress, and curiosity. The rendering may include posting the mental state information to a social network. The method may further comprise collecting mental state data from a second wearer of a second wearable-camera device. The mental state data may be collected for a plurality of people. The wearable-camera device may collect mental state data on the plurality of people. A plurality of wearable-camera devices may be used to collect mental state data. The method may further comprise evaluating a collective mood for the plurality of people. The method may further comprise generating a map showing mental state information across the map. The map may be based on GPS information.

In embodiments, a computer-implemented method for mental state analysis may comprise: receiving mental state information collected from an individual based on a wearable-camera device; analyzing the mental state data to produce mental state information; and sending the mental state information for rendering. In some embodiments, a computer-implemented method for mental state analysis may comprise: collecting mental state data for an individual using a wearable-camera device; analyzing the mental state data to produce mental state information; and sending the mental state information to a server for: further analysis of the mental state information; and rendering a result based on the mental state data. In embodiments, a computer-implemented method for mental state analysis may comprise: receiving an analysis of mental state data which was captured using a wearable-camera device; and rendering an output based on the analysis of the mental state data. In some embodiments, a computer program product embodied in a non-transitory computer readable medium mental state analysis may comprise: code for collecting mental state data using a wearable-camera device; code for analyzing the mental state data to produce mental state information; and code for rendering the mental state information. In embodiments, a computer system for mental state analysis may comprise: a memory which stores instructions; one or more processors attached to the memory wherein the one or more processors, when executing the instructions which are stored, are configured to: collect mental state data using a wearable-camera device; analyze the mental state data to produce mental state information; and render the mental state information. In some embodiments, an apparatus for mental state analysis may comprise: a wearable-camera device wherein the wearable-camera device is on a person; a collector of mental state data wherein the mental state data is received from the wearable-camera device; an analyzer of mental state data that produces mental state information; and a speaker that renders the mental state information to a wearer of the wearable-camera device.

Various features, aspects, and advantages of numerous embodiments will become more apparent from the following description.

### BRIEF DESCRIPTION OF THE DRAWINGS

The following detailed description of certain embodiments may be understood by reference to the following figures wherein:

FIG. 1 is a flow diagram for mental state analysis using a wearable-camera device.

FIG. 2 is a diagram showing use of a wearable-camera device for mental state analysis of another person.

FIG. 3 is a diagram representing camera usage and physiological analysis.

FIG. 4 is a diagram of video and heart related sensing.

FIG. 5 is a system diagram for mental state analysis using a wearable-camera device.

### DETAILED DESCRIPTION

The present disclosure provides a description of various methods and systems for mental state analysis, using a wearable-camera device to evaluate the mental states of a person being viewed. Certain people, such as those with autism spectrum disorders or those with sight limitations, may have trouble recognizing the mental state of someone with whom they are interacting. Thus, such people may have difficulty recognizing anger or confusion in another person as well as other emotions. As the apparatus is wearable, it can analyze a subject as the person wearing the apparatus views the subject. The subject, or person being observed, can then be analyzed and the mental state information be rendered to the viewer wearing the device. Information about the evaluated mental states may be fed back to the viewer wearing the device. The device may include an ear-mounted camera, a glasses mounted camera, a shoulder-mounted camera, a clothing-mounted camera, or other wearable camera. The information may be fed back in the form of audio indicators, tactile indicators, or other means. The wearable-camera device may be used, as a wearer watches another person, to measure mental state data, to collect physiological and actigraphy data, and the like. The mental state data may be used as a gauge for various activities including education, training, assistance, and the like. Such a wearable-camera device may be used to aid visually impaired people, those who are autistic, or those with a visual or learning disability. By using the wearable-camera device, auditory information may be fed to the person wearing the device and may provide information about the other person's facial mental state cues that otherwise may have been missed. Similarly, a tactile cue, such as a vibration, may be used to indicate analysis of a certain mental state. Another application may include obtaining information regarding a collective mental state. The collective mental state may comprise the mental state of a group of people such as employees of a corporation, customers of a company, or citizens of a nation. Geographical information pertaining to the mental state may also be rendered. For example, a map of a nation may indicate regions of the nation that are experiencing collective worry, anger, frustration, happiness, contentedness, or the like. In some embodiments, the scene being viewed by the DWP is recorded and correlated with the mental state of the DWP. Hence, embodiments of the present disclosure are well suited to furthering the study of mental states and the external stimuli that induces those mental states.

Mental state data may be collected for an individual while the person is being viewed by another individual wearing a device. The mental state data may include facial data from a camera. Mental state data may also be collected from the individual doing the viewing by using sensors to collect physiological and actigraphy data. Any or all of the collected mental state data may be analyzed to create mental state information. Mental state information may include moods, mental state data, or other analysis derived or inferred from mental state data. Mental states of the individual being viewed may include frustration, confusion, disappointment, hesitation, cognitive overload, focusing, being engaged, attending, boredom, exploration, confidence, trust, delight, satisfaction, or other emotions or cognitive states. Mental state information may relate to a specific stimulus to which a person may react, such as the actions of another person, a particular web-enabled application, or the like, or may relate to a mood, which may involve a mental state over a relatively longer period of time, such as a person's mental state for a day. Audio indicators may be used to feed information about the mental states of the person being viewed back to the individual doing the viewing.

The mental state data may be stored for later analysis and/or transmitted to a mobile platform. The mental state data may be transmitted to a server. Mental state data received from a server may be used to render mental state information via audio, via a display, or via both audio and a display. Shared and aggregated mental state information may be communicated on a social network.

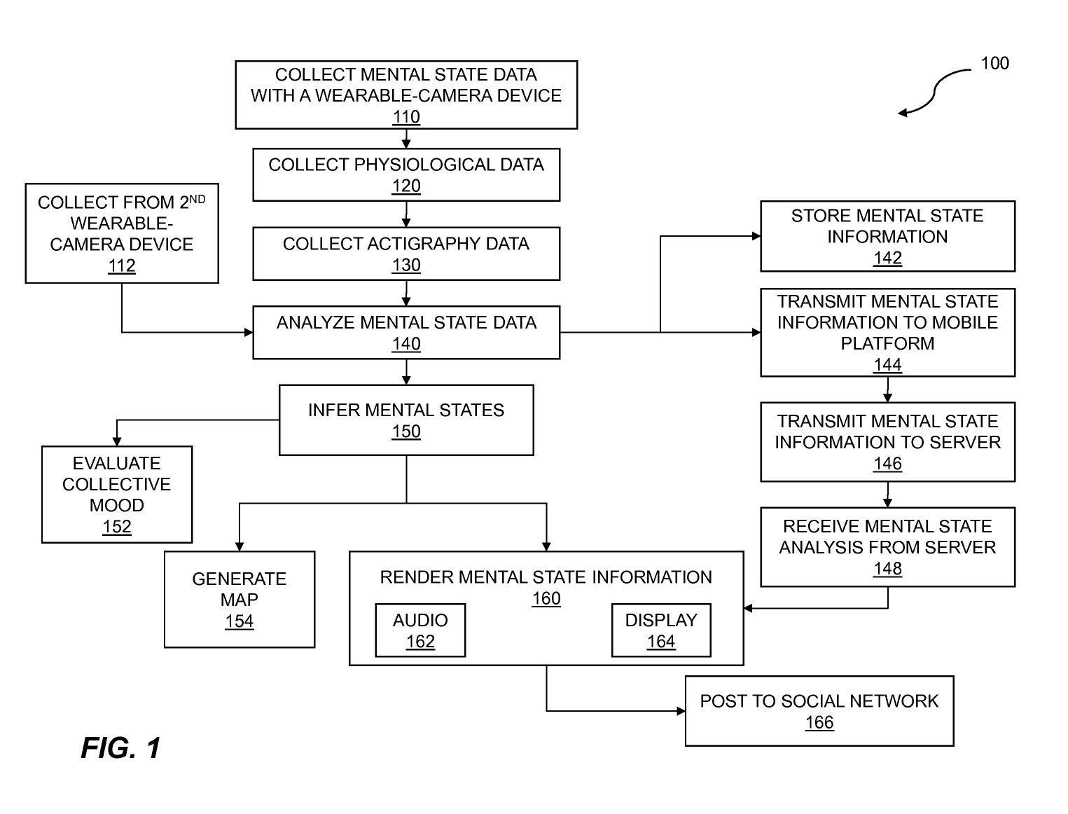

FIG. 1 is a flow diagram for mental state analysis using a wearable-camera device. A flow 100 may begin with collecting mental state data 110 from an individual using a wearable camera device wherein the wearable-camera device includes an ear-mounted camera. The collecting of mental state data may include collecting action units, collecting facial expressions, and the like. A wearable-camera device may be on a person, and the person's head may be pointed at the person on whom mental state analysis is being performed. Mental state data may be collected on a person at whom the wearable-camera device is pointed.

The flow 100 may continue with collecting data from a second wearable-camera device 112 worn by a second person. Embodiments may include collecting mental state data from the second wearer of the wearable-camera device. Embodiments may include collecting mental state data on multiple people wearing wearable-camera devices, where the data from each wearable-camera device may be aggregated to generate collective data, and embodiments may include evaluating a collective mood for the plurality of people. Hence, embodiments may include mental state data that is collected for a plurality of people.

The flow 100 may continue with collecting physiological data 120 which may include one of electrodermal activity, heart rate, heart rate variability, skin temperature, and respiration. For example, heart rate, heart rate variability, autonomic activity, respiration, and perspiration may be observed from video capture. In some embodiments, information on the viewer may be collected using a biosensor to capture physiological information 120 and an accelerometer to capture actigraphy data 130. The types of actigraphy data 130 that may be collected from the person wearing the wearable-camera device may include data pertaining to the human rest/activity cycle, body movement, physical activity levels, and the like.

The collecting of physiological data 120 may be accomplished using a wearable device mounted sensor worn by the observer. The sensor may include, but is not limited to, a heart rate sensor, an electrodermal sensor, and a body temperature sensor. In some embodiments, permission may be requested and obtained prior to the collection of mental state data 110. The flow 100 may continue with analyzing the mental state data 140 to produce mental state information. While mental state data may be raw data such as heart rate, mental state information may include information derived from the raw data. The mental state information may include the mental state data. The mental state information may include valence and arousal. The mental state information may include information on the mental states experienced by the individual doing the viewing or the person being observed. Some embodiments may include the inferring of mental states based on the mental state data which was collected.

The flow 100 may continue with storing mental state information 142 based on the mental state data which was collected. The mental state information may be stored locally within the wearable-camera device, or remotely. Whether stored locally or remotely, the mental state information may be stored on any of a variety of storage devices including Flash, SRAM, DRAM, and the like.

The flow 100 may continue with transmitting the mental state information to a mobile platform 144. Any of a variety of mobile devices may be used as the mobile platform, and the mobile platform may be one of a mobile phone, a tablet computer, a PDA, a laptop, and the like. Transmitting mental state information from a mobile platform to a server may be accomplished by any of a variety of wireless data-transmission techniques including Bluetooth™, Wi-Fi, near field communication (NFC), and the like. Similarly, any of a variety of wired data-transmission techniques may be used to transmit data from the mobile platform to the server, including USB, FireWire™ (IEEE 1394), ThunderBolt™, Ethernet, and the like.

The flow 100 may continue with transmitting the mental state information from the mobile platform to a server 146. Any of a variety of wireless data-transmission techniques may be used to transmit data from the mobile platform to the server. In embodiments, the mental state information may be transmitted from the mobile platform to a server 146 via the Internet.

The flow 100 may continue with receiving mental state analysis from a server 148 based on the mental state information. A server may analyze the mental state data which was transmitted to it. The mental state analysis received from the server may then be rendered by various means.

The flow 100 may include inferring of mental states 150 based on the mental state data which was collected. The mental states may include one of a group consisting of frustration, confusion, disappointment, hesitation, cognitive overload, focusing, engagement, attention, boredom, exploration, confidence, trust, delight, disgust, skepticism, doubt, excitement, laughter, calmness, stress, and curiosity. In embodiments, hybrid analysis may be performed, where some of the analysis is performed on the wearable-camera device, some of the analysis is performed on the mobile platform, some of the analysis is performed on the server, or any combination thereof.

The flow 100 may include evaluating a collective mood 152 based on the mental state data which was collected. This evaluation of a collective mood may include receiving mental state data from multiple DWPs, where each DWP may obtain data for multiple PBOs. The mental state data may be analyzed by the server to derive the collective mood of a group of people. The group can range in size from a small group, such as a team of people or a classroom, to a large group, such as an entire country.

The flow 100 may include generating a map 154 based on the mental state data which was collected. The map may provide a graphical representation of the mental state of a group of people, indicating a geographic position. The map may cover a small area, such as a room, auditorium, stadium, or campus. Alternatively, the map may cover a large area such as a nation or continent. Icons may be used to indicate various mental states (e.g. a “happy face” icon for a happy mental state). Hence, embodiments may include generating a map showing mental state information across the map. In embodiments, the map is based on GPS information.

The flow 100 may include rendering mental state analysis information 160. The rendering may produce audio 162 feedback on the mental state information. The audio feedback may be provided to the wearer of the wearable-camera device. The audio feedback may be in the form of verbal indications about the mental states of the person being viewed. The audio feedback might also comprise tonal indicators. In either case, the audio indicators may suggest the mental states of the person being viewed, including frustration, confusion, disappointment, hesitation, cognitive overload, focusing, engagement, attention, boredom, exploration, confidence, trust, delight, disgust, skepticism, doubt, satisfaction, excitement, laughter, calmness, stress, and curiosity. The rendering may include a display 164 of mental state information. The display may be, but is not limited to, a television monitor, a projector, a computer monitor (including a laptop screen, a tablet screen, a net book screen, and the like), a cell phone display, a mobile device, or another electronic display. In some embodiments, the rendering may include a tactile component, such as a vibrator affixed to the wearable-camera device, to provide an indication to the wearer of a detected mental state. For example, the device may be configured to vibrate when a mental state of anger or worry is detected on the PBO. The flow 100 may include posting the mental state information to a social network 166 as part of the rendering. The social network may provide updates to other members of a user's social network pertaining to the analyzed mental state. Hence, the other members may receive an update such as “Joe seems happy today.” In some embodiments, the social network may offer an action to the other members in response to the analyzed mental state. For example, the other members may receive an update such as “Joe seems sad today, click the link below to send him a message to cheer him up!” In another embodiment, the other members may receive an offer to purchase a gift for the member based on a mental state. For example, the other members may receive an update such as “Jane seems sad today, click the link below to send her some flowers!” Hence, the social network may provide updates, actions, and purchase offers based on inferred or detected mental states. Various steps in the flow 100 may be changed in order, repeated, omitted, or the like without departing from the disclosed concepts. Various embodiments of the flow 100 may be included in a computer program product embodied in a non-transitory computer readable medium that includes code executable by one or more processors.

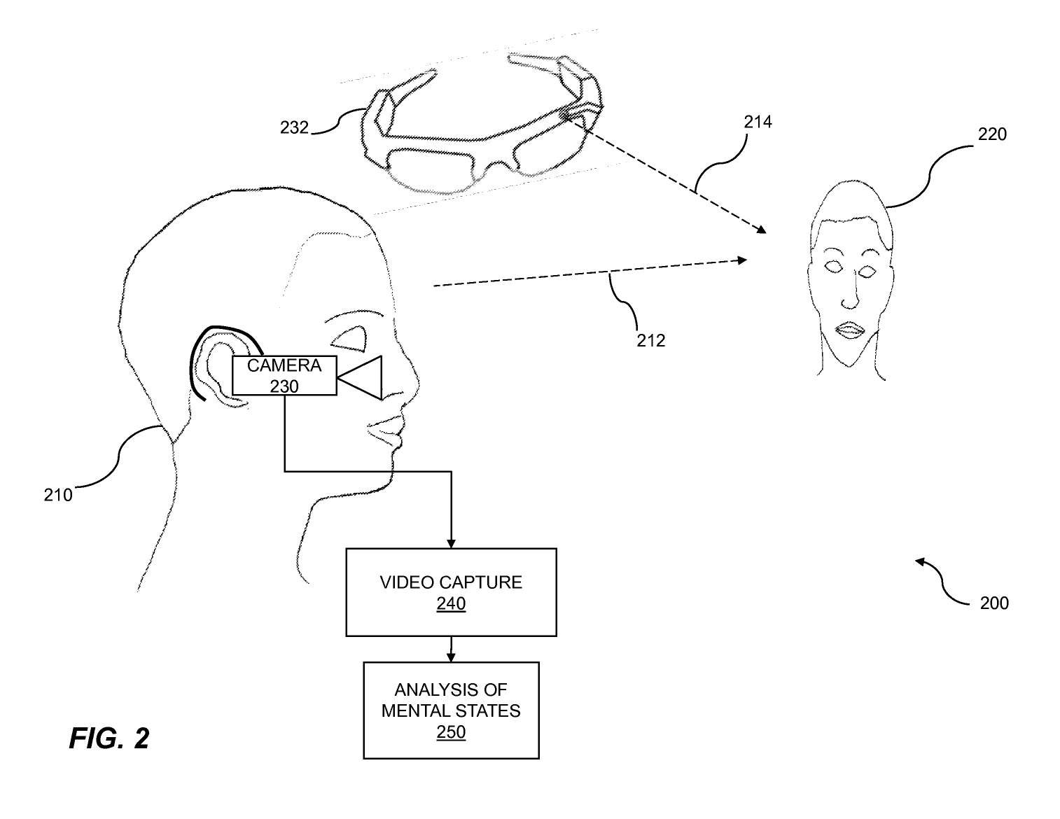

FIG. 2 is a diagram showing use of a wearable-camera device for mental state analysis of another person. In the system 200, one person 210 with a line of sight 212 to another person 220 may wear an ear-mounted camera 230. The first person 210 is referred to as the device-wearing person, and the second person 220 is referred to as the person being observed. The ear-mounted camera 230 may be on a wearer 210, and the wearer's head may be pointed at the same person at whom the camera is pointed. Mental state data may be collected on a person at whom the wearable-camera device is pointed. In embodiments, the wearer may be visually impaired. In embodiments, the wearer may have a non-verbal learning disorder. In embodiments, the wearer may be autistic. The camera 230 may be used to capture one or more of facial data and physiological data. The facial data may include information on facial expressions, action units, head gestures, smiles, brow furrows, squints, lowered eyebrows, raised eyebrows, or attention, in various embodiments. The mental state data collected may comprise physiological data, including one or more of heart rate, heart rate variability, skin temperature, and respiration. In embodiments, the wearable-camera device may include a thermal imaging camera. The heart rate may be ascertained by performing additional image processing on the video of the PBO. For example, captured images of the PBO may be split into red, green, and blue components, where, on a flat surface such as the forehead, a pattern correlating to the PBO's heart rate may be detected. The mental state data collected may include actigraphy data on the viewer. The camera 230 may capture video 240, audio, and/or still images of the PBO 220 into a video capture device. The video capture device may be on the wearable-camera device, on a mobile device (platform), and so on. A camera, as the term is used herein and in the claims, may be a video camera, a still camera, a thermal imager, a CCD device, a three-dimensional camera, a depth camera, or any other type of image-capture apparatus that may allow data captured to be used in an electronic system. In embodiments, the camera 230 may also include a microphone for audio capture. Embodiments may include audio and/or speech analysis performed on the PBO 220. Hence, the tones and language used by the PBO may be analyzed as part of determining a mental state. Furthermore, while the aforementioned embodiments utilize an ear-mounted camera, other embodiments may utilize other means of affixing the camera to a person such as a headband, necklace, lapel clip, or a shirt pocket clip. Similarly, eyeglasses 232, a hat, or other locations may also be utilized as a placement location for a wearable camera. Each of these may view 214 the person being observed 220.

Analysis of mental states 250 is performed using the data captured 240 by the camera 230. The analysis may be performed on the wearable-camera device 230, on a mobile device (platform), or on a server. Analysis may include inferring mental states, where the mental states may include one or more of frustration, confusion, disappointment, hesitation, cognitive overload, focusing, engagement, attention, boredom, exploration, confidence, trust, delight, disgust, skepticism, doubt, satisfaction, excitement, laughter, calmness, stress, and curiosity. Analysis of action units, gestures, and mental states may be accomplished using the captured images of the person 220. The action units may be used to identify smiles, frowns, and other facial indicators of mental states. The gestures, including head gestures, may indicate interest or curiosity. For example, a head gesture of moving toward the person 220 may indicate increased interest or a desire for clarification. Based on the captured images, analysis of physiological data may be performed. Respiration, heart rate, heart rate variability, perspiration, skin temperature, and other physiological indicators of mental state can be observed by analyzing the images. So, in various embodiments, a camera is used to capture one or more of the facial data and the physiological data.

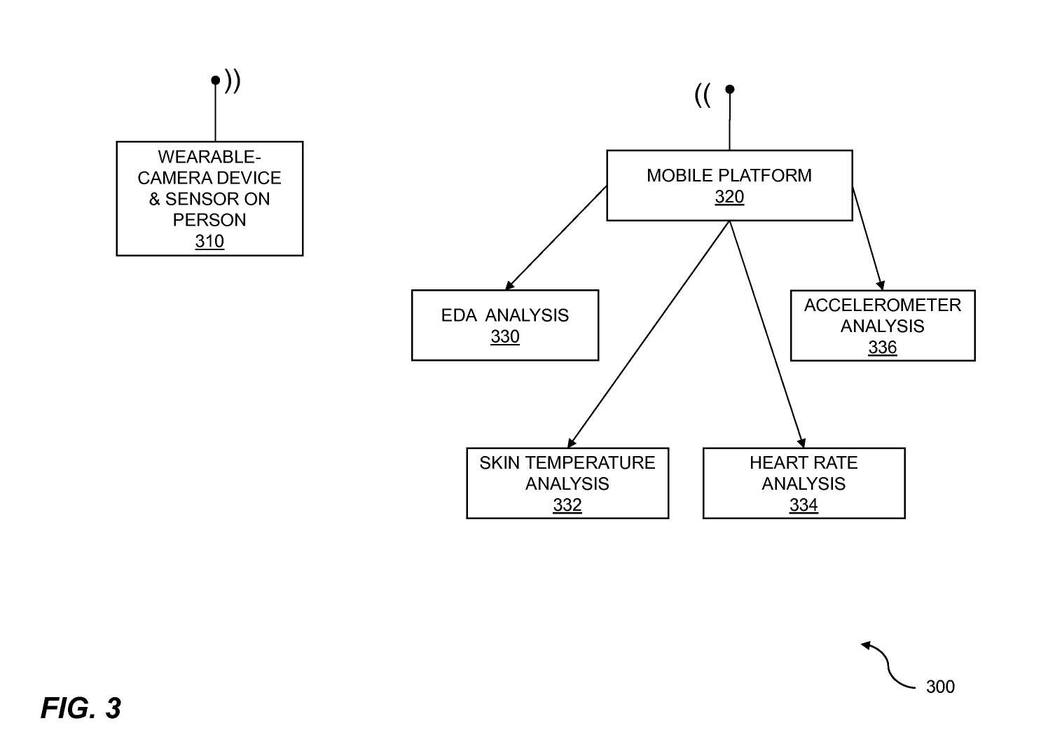

FIG. 3 is a diagram representing camera usage and physiological analysis. A system 300 may analyze a person for whom data is being collected. The person may have a camera and biosensor 310 attached to him or her so that the mental state data can be collected using the camera and biosensor 310. The wearable camera and biosensor 310 may be ear mounted. In other embodiments, the camera and biosensor 310 may be mounted on a headband, necklace, belt, jacket lapel, shirt pocket, or eyeglasses. In some embodiments, additional biosensors may be placed on the body in multiple locations. In some embodiments, sensors may be placed on the person being viewed. The camera and biosensor 310 may include detectors for physiological data, such as electrodermal activity, skin temperature, accelerometer readings, and the like. Other detectors for physiological data may be included as well, such as heart rate, blood pressure, EKG, EEG, other brain waves, and other physiological detectors. The camera and biosensor 310 may transmit information collected to a receiver, such as a mobile platform 320, using wireless technology such as Wi-Fi, Bluetooth, 802.11, cellular, near field communication (NFC), or another band. In other embodiments, the camera and biosensor 310 may communicate with the mobile platform 320 by other methods, such as a wired or optical interface. The mobile platform may provide the data to one or more components in the system 300. In some embodiments, the camera and biosensor 310 may record various types of physiological information in memory for later download and analysis. In some embodiments, the download of data representing the recorded physiological information may be accomplished through a USB port or another form of wired or wireless connection. The collecting of physiological data may be accomplished using a sensor that is mounted on a person on whom the mental state data is being collected.

Mental states may be inferred based on physiological data, such as physiological data obtained from the camera and biosensor 310. Mental states may also be inferred based on facial expressions and head gestures observed by a camera, or based on a combination of data from the camera and the biosensor 310. The mental states may be analyzed based on arousal and valence. Arousal can range from being highly activated, such as when someone is agitated, to being entirely passive, such as when someone is bored. Valence can range from being very positive, such as when someone is happy, to being very negative, such as when someone is angry. Physiological data may include electrodermal activity (EDA), skin conductance, accelerometer readings, skin temperature, heart rate, and heart rate variability, along with other types of analysis of a human being. It will be understood that both here and elsewhere in this document, some physiological information can be obtained by a camera and biosensor 310. Facial data may include facial actions and head gestures used to infer mental states. Further, the data may include information on hand gestures, body language, and body movements such as visible fidgets. In some embodiments, such movements may be captured by cameras or by sensor readings. Facial data may include a measurement of head tilting, leaning forward, smiling, frowning, as well as many other gestures or expressions. In some embodiments, audio data may also be collected and analyzed for the purposes of inferring mental states. The audio data may include, but is not limited to, volume, frequency, and dynamic range of tones. In some embodiments, language analysis may also be performed and used for the purposes of inferring mental states.

Electrodermal activity may be collected and analyzed 330. In some embodiments the electrodermal activity may be collected continuously, every second, four times per second, eight times per second, 32 times per second, or on some other periodic basis. The electrodermal activity may be recorded. The recording may be to a disk, a tape, onto flash memory, into a computer system, or streamed to a server. The electrodermal activity may be analyzed 330 to indicate arousal, excitement, boredom, or other mental states based on changes in skin conductance. Skin temperature may be collected on a periodic basis and may be recorded. The skin temperature may be analyzed 332 and may indicate arousal, excitement, boredom, or other mental states based on changes in skin temperature. The heart rate may be collected and recorded. The heart rate may be analyzed 334 and a high heart rate may indicate excitement, arousal, or other mental states. Accelerometer data may be collected and may indicate one, two, or three dimensions of motion. The accelerometer data may be recorded. The accelerometer data may be used to create an actigraph showing an individual's activity level over time. The accelerometer data may be analyzed 336 and may indicate a sleep pattern, a state of high activity, a state of lethargy, or another state based on accelerometer data.

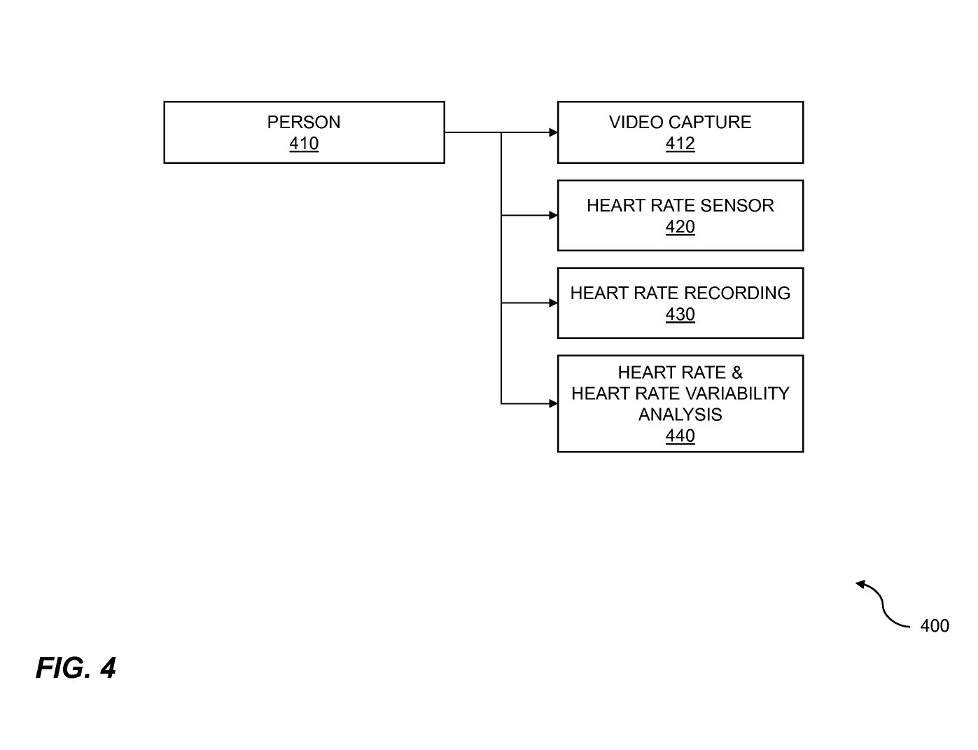

FIG. 4 is a diagram of video and heart-related sensing. In the diagram a person 410 is observed by a system 400 which may include video capture 412 with a wearable-camera device. A camera, as the term is used herein and in the claims, may be a video camera, a still camera, a thermal imager, a CCD device, a three-dimensional camera, a depth camera, or any other type of image-capture apparatus that may allow data captured to be used in an electronic system. A heart rate sensor 420, a specific type of biosensor, may further analyze a person 410. The observation may be through a contact sensor or through contactless sensing including, but not limited to, video analysis to capture heart rate information. In some embodiments, a webcam is used to capture the physiological data. In some embodiments, the physiological data is used to determine autonomic activity, and the autonomic activity may be one of a group comprising heart rate, respiration, and heart rate variability. Other embodiments may determine other autonomic activity such as pupil dilation. The heart rate may be recorded 430 to a disk or a tape, placed into flash memory or a computer system, or streamed to a server. The heart rate and heart rate variability may be analyzed 440. An elevated heart rate may indicate excitement, nervousness, or other mental states. A lowered heart rate may indicate calmness, boredom, or other mental states. The level of heart-rate variability may be associated with fitness, calmness, stress, and age. The heart-rate variability may be used to help infer the mental state. High heart-rate variability may indicate good health and lack of stress. Low heart-rate variability may indicate an elevated level of stress. Furthermore, the heart-rate variability may also indicate a level of engagement in external stimuli. For example, high heart-rate variability may be associated with high levels of mental engagement in external stimuli, whereas low heart-rate variability may be associated with a subject who is not very engaged, and may not be very interested in external stimuli. Thus, physiological data may include one or more of electrodermal activity, heart rate, heart rate variability, skin temperature, and respiration.

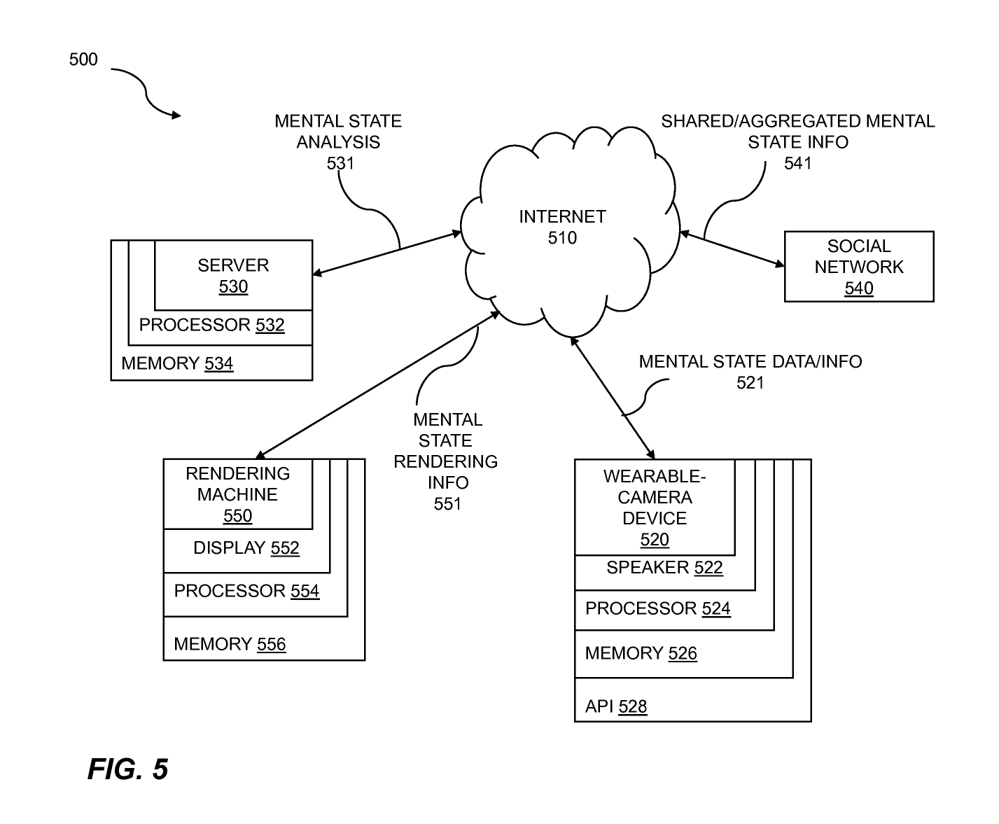

FIG. 5 is a system diagram for a system 500 for mental state analysis using a wearable-camera device. The Internet 510, intranet, or another computer network may be used for communication between the various devices. A wearable device with a camera 520 has a memory 526 for storing instructions and one or more processors 524 connected to the memory 526 wherein the one or more processors 524 can execute instructions. The wearable-camera device 520 also may have wired or wireless connections to carry mental state information 521, and a speaker 522 that may present various audio renderings to a user. The wearable-camera device 520 can include an application programming interface (API) 528. The API 528 can provide a protocol for software components to interface with the wearable-camera device 520. The software components may be provided by third parties and control and use certain aspects of the wearable-camera device 520. A library of software components or plug-in routines may be used to aid in mental state analysis and provide emotion enablement for the wearable-camera device 520. The wearable-camera device 520 may be able to collect mental state data from an individual or a plurality of people as they view another person or plurality of people. In some embodiments, there may be multiple wearable-camera devices 520 that each may collect mental state data from one person or a plurality of people as they interact with a person or people. The wearable-camera device 520 may communicate with the server 530 over the Internet 510, another computer network, or by any other method suitable for communication between two computers. In some embodiments, the server 530 functionality may be embodied in the wearable-camera device 520.

The server 530 may have an internet connection for receiving mental states or collected mental state information 531, have a memory 534 which stores instructions, and may have one or more processors 532 attached to the memory 534 to execute instructions. The server 530 may receive, from the wearable device or devices with cameras 520, mental state information 521 collected from a plurality of people as they view a person or persons. The server 530 may analyze the mental state data to produce mental state information. The server 530 may also aggregate mental state information on the plurality of people who view a person or persons. The server 530 may associate the aggregated mental state information with a rendering and also with a collection of norms for the context being measured.

In some embodiments, the server 530 may also allow users to view and evaluate the mental state information that is associated with the viewing of a person or persons. In other embodiments, the server 530 may send the shared and/or aggregated mental state information 541 to a social network 540 to be shared, distributing the mental state information across a computer network. In some embodiments, the social network 540 may run on the server 530.

The system 500 may include a rendering machine 550. The rendering machine may include one or more processors 554 coupled to a memory 556 to store instructions and a display 552. The rendering machine 550 may receive the mental state rendering information 551 from the Internet 510 or another computer-aided communication method. The mental state rendering information 551 may include mental state analysis from the server 530, shared/aggregated mental state information 541 from the social network 540, or mental state data/information 521 from the wearable-camera device 520. Related output may be rendered to a display 552. The display may comprise, but is not limited to, a television monitor, a projector, a computer monitor (including a laptop screen, a tablet screen, a net book screen, and the like), a cell phone display, a mobile device, or another electronic display.

The system 500 may include a computer program product embodied in a non-transitory computer readable medium for mental state analysis, the computer program product comprising: code for collecting mental state data using wearable-camera device, code for analyzing the mental state data to produce mental state information, and code for rendering the mental state information. In embodiments, the system 500 for mental state analysis may include a memory which stores instructions and one or more processors attached to the memory wherein the one or more processors when executing the instructions which are stored, are configured to: collect mental state data using an wearable-camera device; analyze the mental state data to produce mental state information; and render the mental state information. In embodiments, the system 500 for mental state analysis may include a wearable-camera device on a person; a collector of mental state data wherein the mental state data is received from the wearable-camera device; an analyzer of mental state data that produces mental state information; and a speaker that renders the mental state information to the wearer of the wearable-camera device. In embodiments, the system 500 may perform a computer-implemented method for mental state analysis comprising: receiving mental state information collected from an individual based on a wearable-camera device; analyzing the mental state data to produce mental state information; and sending the mental state information for rendering. In embodiments, the system 500 may perform a computer-implemented method for mental state analysis comprising: collecting mental state data for an individual using a wearable-camera device; analyzing the mental state data to produce mental state information; and sending the mental state information to a server for: further analysis of the mental state information; and rendering a result based on the mental state data. In embodiments, the system 500 may perform a computer-implemented method for mental state analysis comprising: receiving an analysis of mental state data which was captured using a wearable-camera device; and rendering an output based on the analysis of the mental state data.

Each of the above methods may be executed using one or more processors on one or more computer systems. Embodiments may include various forms of distributed computing, client/server computing, and cloud based computing. Further, it will be understood that for each flow chart in this disclosure, the depicted steps or boxes are provided for purposes of illustration and explanation only. The steps may be modified, omitted, or re-ordered and other steps may be added without departing from the scope of this disclosure. Further, each step may contain one or more sub-steps. While the foregoing drawings and description set forth functional aspects of the disclosed systems, no particular arrangement of software and/or hardware for implementing these functional aspects should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. Each element of the block diagrams and flowchart illustrations, as well as each respective combination of elements in the block diagrams and flowchart illustrations, illustrates a function, step or group of steps of the methods, apparatus, systems, computer program products and/or computer-implemented methods. Any and all such functions may be implemented by computer program instructions, by special-purpose hardware-based computer systems, by combinations of special purpose hardware and computer instructions, by combinations of general purpose hardware and computer instructions, and so on. Any and all of which may be generally referred to herein as a “circuit,” “module,” or “system.”

A programmable apparatus which executes any of the above mentioned computer program products or computer implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are not limited to applications involving conventional computer programs or programmable apparatus that run them. It is contemplated, for example, that embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized. The computer readable medium may be a non-transitory computer readable medium for storage. A computer readable storage medium may be electronic, magnetic, optical, electromagnetic, infrared, semiconductor, or any suitable combination of the foregoing. Further computer readable storage medium examples may include an electrical connection having one or more wires, a portable computer diskette, a hard disk, a random access memory (RAM), a read-only memory (ROM), an erasable programmable read-only memory (EPROM, Flash, MRAM, FeRAM, or phase change memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. Each thread may spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the entity causing the step to be performed.

While the invention has been disclosed in connection with preferred embodiments shown and described in detail, various modifications and improvements thereon will become apparent to those skilled in the art. Accordingly, the spirit and scope of the present invention is not to be limited by the foregoing examples, but is to be understood in the broadest sense allowable by law.

What is claimed is:

1. A computer-implemented method for mental state analysis comprising:
collecting mental state data using a wearable-camera device wherein the wearable-camera device includes an ear-mounted camera;
analyzing the mental state data to produce mental state information; and
rendering the mental state information.

2-3. (canceled)

4. The method of claim 1 wherein the rendering produces audio feedback on the mental state information.

5. The method of claim 4 wherein the audio feedback is provided to a wearer of the wearable-camera device.

6-9. (canceled)

10. The method of claim 1 wherein the collecting mental state data further comprises collecting physiological data including one of electrodermal activity, heart rate, heart rate variability, skin temperature, and respiration.

11. The method of claim 10 wherein the collecting of physiological data is accomplished using a sensor that is mounted on a person on whom the mental state data is being collected.

12. The method of claim 1 wherein the collecting of mental state data further comprises actigraphy data.

13. (canceled)

14. The method of claim 1 wherein the mental state information is transmitted to a mobile platform.

15. The method of claim 14 wherein the mobile platform is one of a mobile phone, a tablet computer, or a mobile device.

16. The method of claim 14 wherein the mental state information is transmitted from the mobile platform to a server.

17. The method of claim 16 further comprising receiving mental state analysis from a server based on the mental state information.

18. The method of claim 17 wherein the rendering is based on the mental state analysis received from the server.

19. The method of claim 1 further comprising inferring mental states based on the mental state data which was obtained wherein the mental states include one or more of frustration, confusion, disappointment, hesitation, cognitive overload, focusing, engagement, attention, boredom, exploration, confidence, trust, delight, disgust, skepticism, doubt, satisfaction, excitement, laughter, calmness, stress, and curiosity.

20. The method of claim 1 wherein the rendering includes posting the mental state information to a social network.

21. The method of claim 1 further comprising collecting mental state data from a second wearer of a second wearable-camera device.

22. The method of claim 1 wherein the mental state data is collected for a plurality of people.

23. The method of claim 22 wherein the wearable-camera device collects mental state data on the plurality of people.

24. The method of claim 22 wherein a plurality of wearable-camera devices are used to collect mental state data.

25. The method of claim 22 further comprising evaluating a collective mood for the plurality of people.

26. The method of claim 22 further comprising generating a map showing mental state information across the map.

27. The method of claim 26 wherein the map is based on GPS information.

28-30. (canceled)

31. A computer program product embodied in a non-transitory computer readable medium mental state analysis, the computer program product comprising:
code for collecting mental state data using a wearable-camera device wherein the wearable-camera device includes an ear-mounted camera;
code for analyzing the mental state data to produce mental state information; and
code for rendering the mental state information.

32. A computer system for mental state analysis comprising:
a memory which stores instructions;
one or more processors attached to the memory wherein the one or more processors, when executing the instructions which are stored, are configured to:
collect mental state data using a wearable-camera device wherein the wearable-camera device includes an ear-mounted camera;
analyze the mental state data to produce mental state information; and
render the mental state information.

33. (canceled)
